(19) United States
(12) Plant Patent Application Publication
(10) Pub. No.: US 2005/0022279 P1
(43) Pub. Date: Jan. 27, 2005

(54) Osteospermum plant named 'Sunny Mary'

(50) OSTEOSPERMUM ECKLONIS; OSTEOSPERMUM

(75) Inventor: Bjarne Nyholm Larsen, Odense N (DK)

Correspondence Address: FOLEY AND LARDNER;SUITE 500, 3000 K STREET NW, WASHINGTON, DC, 20007, US

(73) Assignee: SUNNY Osteospermum ApS

(21) Appl. No.: 10/873,228

(22) Filed: Jun. 23, 2004

(30) Foreign Application Priority Data
Jul. 7, 2003 (QZ) 2003/1239

(51) Int. Cl. A01H 5/00

(52) U.S. Cl. PLT/360

(57) ABSTRACT

A new distinct cultivar of Osteospermum plant named ‘Sunny Mary’, characterized by its large upright purple flower heads, flower colors: Ray florets from RHS 71A red-purple with stripes of RHS 59A purple to RHS 67A red-purple with stripes of RHS 72A; dense and bushy plant form, mainly due to more upright stems; and compact plant habit; moderately vigorous growth habit, but less need for chemical growth retardation; and fewer and smaller flowers per plant.

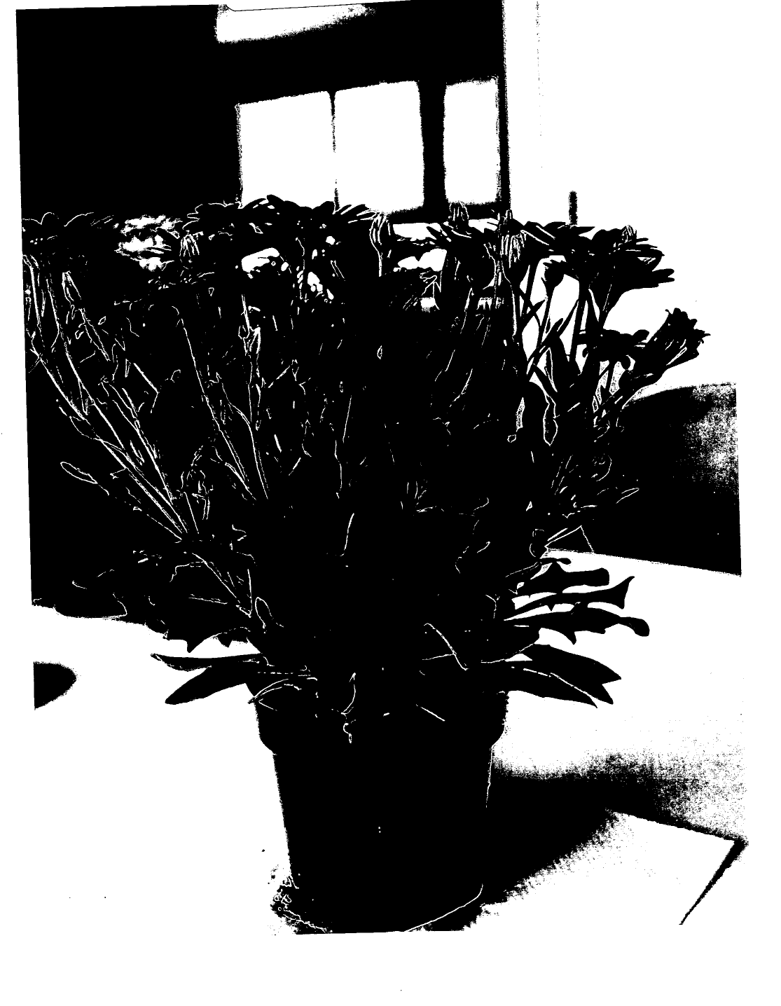

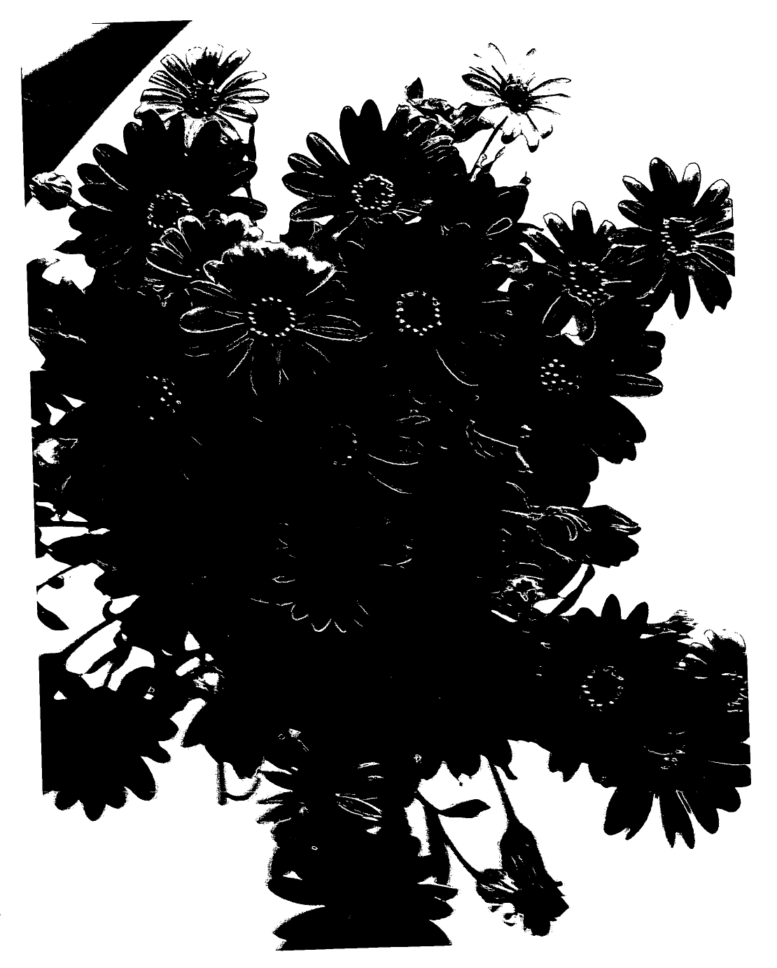

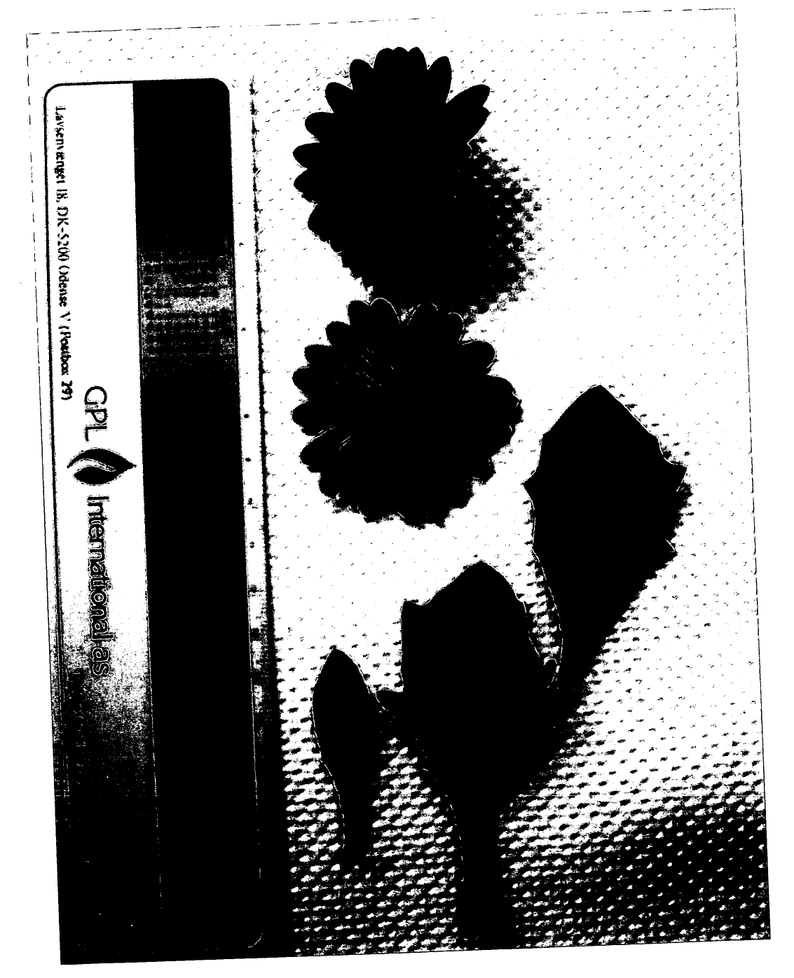

### GENUS AND SPECIES OF THE PLANT CLAIMED

Osteospermum eclonis (DC) T. Norl.

### VARIETY DENOMINATION

Sunny Mary

### BACKGROUND OF THE INVENTION

The present Invention relates to a new and distinct cultivar of Osteospermum plant, botanically known as Osteospermum eclonis (DC) T. Norl., commonly known as Cape Daisy and hereinafter referred to by the variety denomination ‘Sunny Mary’.

The new Osteospermum is a product of a planned breeding program conducted by the Inventor, Bjarne Larsen, in Stige, Denmark. The new Osteospermum originated from a cross made in 2000 by the Inventor between Osteospermum eclonis (DC) T. Norl. seedling designated ‘9913’ and Osteospermum eclonis (DC) T. Norl. seedling designated ‘90.018.00’. The Inventor selected the new Osteospermum cultivar from the progeny of the above crossing in 2001 on the basis of its flower color and compact, freely branching habit. Plants of the new Osteospermum are upright, compact and have a unique color.

Asexual reproduction of the new cultivar by terminal cuttings taken and propagated in trial production batches in Stige, Denmark, has shown that the unique features of this new Osteospermum are stable and reproduced true to type in many successive generations.

### SUMMARY OF THE INVENTION

The following traits have been repeatedly observed and are determined to be the unique characteristics of ‘Sunny Mary’. These characteristics in combination distinguish ‘Sunny Mary’ as a new and distinct cultivar:

1. Large upright purple flower heads, flower colors: Ray florets from RHS 71A red-purple with stripes of RHS 59A purple to RHS 67A red-purple with stripes of RHS 72A;

2. dense and bushy plant form, mainly due to more upright stems; and compact plant habit;

3. moderately vigorous growth habit, but less need for chemical growth retardation; and

4. fewer and smaller flowers per plant.

Plants of the cultivar ‘Sunny Mary’ can be compared to plants of the Osteospermum eclonis (DC) T. Norl. Cultivar ‘Sunny Nathalie’ (unpatented). However, in side-by-side comparisons conducted by the Inventor in Stige Denmark, plants of the cultivar ‘Sunny Mary’ and the cultivar ‘Sunny Nathalie’ differ in the following characteristics:

1. Plants of the new Osteospermum have striking color combinations of deep purple to red ray flowers and blue disk with orange pollen.

2. Plants of the new Osteospermum have fewer leaves and longer internodes than plants of the cultivar ‘Sunny Stephanie’.

3. Plants of the new Osteospermum have shorter and thinner peduncles than plants of the cultivar ‘Sunny Stephanie’.

4. Plants of the new Osteospermum are shorter and more compact than the plants of the cultivar ‘Sunny Stephanie’

5. Plants of the new Osteospermum have less, but smaller flowers per plant than the plants of the cultivar ‘Sunny Stephanie’

Plants of the cultivar ‘Sunny Mary’ have not been observed under all possible environmental conditions. The phenotype may vary somewhat with variations in environment such as temperature, light intensity, day length, and fertility level without, however, any variance in genotype.

### BRIEF DESCRIPTION OF THE PHOTOGRAPHS

The accompanying color photographs illustrate the overall appearance and details of flower form color and structures of the new cultivar, showing the colors as true as it is reasonably possible to obtain in color reproductions of this type. Colors in the photographs may differ slightly from the color values cited in the detailed botanical description, which more accurately describe the actual colors of the new Osteospermum. The first photograph shows a side perspective view of a typical flowering plant of ‘Sunny Mary’ as grown in an 11 cm pot. The second photo is a close-up, top view of the composite young and older flowers. The third photograph shows the detail of front and back of a ‘Sunny Mary’ flower as well as the front and back of the leaf.

### DETAILED BOTANICAL DESCRIPTION

In the following description, color references are made to The Royal Horticultural Society Colour Chart (RHS), 4^{th} edition. Plants were grown under greenhouse conditions. Plants used for this description were grown for about 20 weeks after cutting.
Botanical classification: Osteospermum eclonis (DC) T. Norl.
Parentage:
   Female parent.—Osteospermum eclonis (DC) T. Norl. Seedling no. ‘9913’.
   Male parent.—Osteospermum eclonis (DC) T. Norl., Seedling no. ‘90.018.00’.
   Propagation.—Type cutting: terminal vegetative cuttings.
   Time to initiate roots.—About 10 to 14 days at 18 to 21C in tunnels in a greenhouse.
   Root description.—Fine, well branched.
Plant description:
   Form.—Perennial plant with upright plant habit. Osteospermum flowers in composite heads. Freely branching with lateral flowering branches forming at every node; dense and bushy.
   Crop time.—After rooting, about 18 weeks are required to produce finished flowering plants in 11 cm pots.
   Plant height (soil level to top of plant plane).—About 18 cm.
   Plant diameter.—18 cm.
   Vigor.—Moderately vigorous growth rate.
Foliage description:
   Leaves.—alternate, single, brochidodromus venation. Length: 4-8 cm. Width: About 30 mm. Shape: Obovate. Apex: obtuse. Apical leaves: acute Base: attenuate to decurrent. Margin: broadly lobed. Texture: smooth, glabrous, shiny. Scattered short, stiff hairs. Color: Young foliage, upper and lower surfaces: 147B, yellow-green. Mature foliage, upper surface N147A, mature foliage lower surface 148A. Venation, 148B.
Flower description:
   Flower arrangement and shape.—Tubular disk and petalled (ligulate) ray flowers in composite daisy capitulae; with 15 parted small involucre. Thin (2 mm) peduncles.
   Natural flowering season.—Continuous throughout the spring and summer in temperate regions. Season can be extended by vernalization and long day treatments.
   Flower longevity on the plant.—5 to 9 days (longevity of individual flowers is highly dependent on temperature and light conditions). Flowers persistent, petals folding and withering slowly to 79A, dark purple.
   Inflorescence diameter.—About 5 cm.
   Flowers.—Length: Ray petals: About 20 mm. Width: Maximum about 78 mm. Elliptic, acute ray petal lobes. Ray petal color: Upper surfaces, 71 A to 67A purple to almost red with stripes of 59A to 72A red-purple. Under surfaces: N81A, Purple-violet to N79A with stripes of N79C. Disk flowers tips N89B, violet-blue, basal tube 75D light violet to 155A white.
Reproductive organs:
   Anthers.—Linear fused, stamen color 202A, black.
   Pollen.—26A orange.
   Pistil and stigma.—16C yellow-orange.
   Peduncle.—Strength: Moderately strong. Length: About 5 cm. Diameter: About 2 mm.
   Color.—144C yellow-green.
   Seed.—Length: About 1 mm. Diameter: About 0.3 mm.
   Weather tolerance.—Plants of the new Osteospermum have exhibited good tolerance to draught, rain and wind, however flowering may cease during hot periods (temperatures above 30 C).

1. A new and distinct cultivar of Osteospermum plant named ‘Sunny Mary’, as illustrated and described herein.
